(19) United States
(12) Patent Application Publication
(10) Pub. No.: US 2005/0107783 A1
(43) Pub. Date: May 19, 2005

(54) Devices for applying energy to tissue

(75) Inventors: Curtis P. Tom, San Mateo, CA (US); Thomas A. Kramer, San Carlos, CA (US); Ed Roschak, Mission Viejo, CA (US); Don Tanaka, Saratoga, CA (US); David Thompson, San Jose, CA (US)

Correspondence Address: BRONCUS TECHNOLOGIES, INC.;BUILDING A8, 1400 N. SHORELINE BLVD., MOUNTAIN VIEW, CA, 94043, US

(73) Assignee: Broncus Technologies, Inc., Mountain View, CA (US)

(21) Appl. No.: 11/015,531

(22) Filed: Dec. 17, 2004

Related U.S. Application Data
(63) Continuation of application No. 10/280,851, filed on Oct. 25, 2002.
Application No. 10/280,851 is a continuation-in-part of application No. 10/080,344, filed on Feb. 21, 2002.
Application No. 10/080,344 is a continuation-in-part of application No. 09/946,706, filed on Sep. 4, 2001, now Pat. No. 6,749,606.
Application No. 10/080,344 is a continuation-in-part of application No. 09/633,651, filed on Aug. 7, 2000, now Pat. No. 6,692,494.
(60) Provisional application No. 60/269,130, filed on Feb. 14, 2001.
(60) Provisional application No. 60/147,528, filed on Aug. 5, 1999.
(60) Provisional application No. 60/176,141, filed on Jan. 14, 2000.

(51) Int. Cl. A61B 18/18

(52) U.S. Cl. 606/47

(57) ABSTRACT

Disclosed herein are devices for altering gaseous flow within a lung to improve the expiration cycle of an individual, particularly individuals having chronic obstructive pulmonary disease (COPD). More particularly, a medical catheter is disclosed to detect the presence of blood vessels and to produce collateral openings or channels through the airway wall so that air is able to pass directly out of the lung tissue to facilitate both the exchange of oxygen ultimately into the blood and/or to decompress hyper-inflated lungs.

### CROSS REFERENCE TO RELATED APPLICATIONS

This application is a continuation of U.S. application Ser. No. 10/280,851 filed Oct. 25, 2002, which is a continuation in part of U.S. application Ser. No. 10/080,344, filed Feb. 21, 2002, which is a continuation in part of U.S. application Ser. No. 09/946,706, filed Sep. 4, 2001, which claims the benefit of U.S. Provisional Application No. 60/269,130, filed on Feb. 14, 2001. U.S. application Ser. No. 09/946,706, filed Sep. 4, 2001, is a continuation in part of U.S. application Ser. No. 09/633,651, filed Aug. 7, 2000, which claims the benefit of U.S. Provisional Application No. 60/147,528, filed on Aug. 5, 1999, and U.S. Provisional Application No. 60/176,141, filed on Jan. 14, 2000. Each of the above referenced applications is incorporated herein by reference in its entirety.

### FIELD OF THE INVENTION

The invention is directed to devices for altering gaseous flow within a lung to improve the expiration cycle of an individual, particularly individuals having chronic obstructive pulmonary disease (COPD). More particularly, devices are disclosed to produce collateral openings or channels through the airway wall so that expired air is able to pass directly out of the lung tissue to facilitate both the exchange of oxygen ultimately into the blood and/or to decompress hyper-inflated lungs.

### BACKGROUND OF THE INVENTION

Chronic obstructive pulmonary disease (COPD) includes emphysema and chronic bronchitis. These diseases are characterized by obstruction to air flow. According to the American Lung Association (ALA), COPD is the fourth leading cause of death, claiming the lives of 119,524 Americans annually. The ALA also states that the annual cost to the United States of America for COPD is approximately $30.4 billion, including healthcare expenditures of $14.7 billion and indirect costs of $15.7 billion.

Those inflicted with COPD face disabilities due to the limited pulmonary functions. Usually, individuals afflicted by COPD also face loss in muscle strength and an inability to perform common daily activities. Often, those patients desiring treatment for COPD seek a physician at a point where the disease is advanced. Since the damage to the lungs is irreversible, there is little hope of recovery. Most times, the physician cannot reverse the effects of the disease but can only offer treatment and advice to halt the progression of the disease.

To understand the detrimental effects of COPD, the workings of the lungs requires a cursory discussion. The primary function of the lungs is to permit the exchange of two gasses by removing carbon dioxide from arterial blood and replacing it with oxygen. Thus, to facilitate this exchange, the lungs provide a blood gas interface. The oxygen and carbon dioxide move between the gas (air) and blood by diffusion. This diffusion is possible since the blood is delivered to one side of the blood-gas interface via small blood vessels (capillaries). The capillaries are wrapped around numerous air sacs called alveoli which function as the blood-gas interface. A typical human lung contains about 300 million alveoli.

The air is brought to the other side of this blood-gas interface by a natural respiratory airway, hereafter referred to as a natural airway or airway, consisting of branching tubes which become narrower, shorter, and more numerous as they penetrate deeper into the lung. Specifically, the airway begins with the trachea which branches into the left and right bronchi which divide into lobar, then segmental bronchi. Ultimately, the branching continues down to the terminal bronchioles which lead to the alveoli. Plates of cartilage may be found as part of the walls throughout most of the airway from the trachea to the bronchi. The cartilage plates become less prevalent as the airways branch. Eventually, in the last generations of the bronchi, the cartilage plates are found only at the branching points. The bronchi and bronchioles may be distinguished as the bronchi lie proximal to the last plate of cartilage found along the airway, while the bronchiole lies distal to the last plate of cartilage. The bronchioles are the smallest airways that do not contain alveoli. The function of the bronchi and bronchioles is to provide conducting airways that lead air to and from the gas-blood interface. However, these conducting airways do not take part in gas exchange because they do not contain alveoli. Rather, the gas exchange takes place in the alveoli which are found in the distal most end of the airways.

The mechanics of breathing include the lungs, the rib cage, the diaphragm and abdominal wall. During inspiration, inspiratory muscles contract increasing the volume of the chest cavity. As a result of the expansion of the chest cavity, the pleural pressure, the pressure within the chest cavity, becomes sub-atmospheric. Consequently, air flows into the lungs and the lungs expand. During unforced expiration, the inspiratory muscles relax and the lungs begin to recoil and reduce in size. The lungs recoil because they contain elastic fibers that allow for expansion, as the lungs inflate, and relaxation, as the lungs deflate, with each breath. This characteristic is called elastic recoil. The recoil of the lungs causes alveolar pressure to exceed atmospheric pressure causing air to flow out of the lungs and deflate the lungs. If the lungs' ability to recoil is damaged, the lungs cannot contract and reduce in size from their inflated state. As a result, the lungs cannot evacuate all of the inspired air.

In addition to elastic recoil, the lung's elastic fibers also assist in keeping small airways open during the exhalation cycle. This effect is also known as “tethering” of the airways. Such tethering is desirable since small airways do not contain cartilage that would otherwise provide structural rigidity for these airways. Without tethering, and in the absence of structural rigidity, the small airways collapse during exhalation and prevent air from exiting thereby trapping air within the lung.

Emphysema is characterized by irreversible biochemical destruction of the alveolar walls that contain the elastic fibers, called elastin, described above. The destruction of the alveolar walls results in a dual problem of reduction of elastic recoil and the loss of tethering of the airways. Unfortunately for the individual suffering from emphysema, these two problems combine to result in extreme hyperinflation (air trapping) of the lung and an inability of the person to exhale. In this situation, the individual will be debilitated since the lungs are unable to perform gas exchange at a satisfactory rate.

One further aspect of alveolar wall destruction is that the airflow between neighboring air sacs, known as collateral ventilation or collateral air flow, is markedly increased as when compared to a healthy lung. While alveolar wall destruction decreases resistance to collateral ventilation, the resulting increased collateral ventilation does not benefit the individual since air is still unable to flow into and out of the lungs. Hence, because this trapped air is rich in CO₂, it is of little or no benefit to the individual.

Chronic bronchitis is characterized by excessive mucus production in the bronchial tree. Usually there is a general increase in bulk (hypertrophy) of the large bronchi and chronic inflammatory changes in the small airways. Excessive amounts of mucus are found in the airways and semisolid plugs of this mucus may occlude some small bronchi. Also, the small airways are usually narrowed and show inflammatory changes.

Currently, although there is no cure for COPD, treatment includes bronchodilator drugs, and lung reduction surgery. The bronchodilator drugs relax and widen the air passages thereby reducing the residual volume and increasing gas flow permitting more oxygen to enter the lungs. Yet, bronchodilator drugs are only effective for a short period of time and require repeated application. Moreover, the bronchodilator drugs are only effective in a certain percentage of the population of those diagnosed with COPD. In some cases, patients suffering from COPD are given supplemental oxygen to assist in breathing. Unfortunately, aside from the impracticalities of needing to maintain and transport a source of oxygen for everyday activities, the oxygen is only partially functional and does not eliminate the effects of the COPD. Moreover, patients requiring a supplemental source of oxygen are usually never able to return to functioning without the oxygen.

Lung volume reduction surgery is a procedure which removes portions of the lung that are over-inflated. The improvement to the patient occurs as a portion of the lung that remains has relatively better elastic recoil which allows for reduced airway obstruction. The reduced lung volume also improves the efficiency of the respiratory muscles. However, lung reduction surgery is an extremely traumatic procedure which involves opening the chest and thoracic cavity to remove a portion of the lung. As such, the procedure involves an extended recovery period. Hence, the long term benefits of this surgery are still being evaluated. In any case, it is thought that lung reduction surgery is sought in those cases of emphysema where only a portion of the lung is emphysematous as opposed to the case where the entire lung is emphysematous. In cases where the lung is only partially emphysematous, removal of a portion of emphysematous lung which was compressing healthier portions of the lung allows the healthier portions to expand, increasing the overall efficiency of the lung. If the entire lung is emphysematous, however, removal of a portion of the lung removes gas exchanging alveolar surfaces, reducing the overall efficiency of the lung. Lung volume reduction surgery is thus not a practical solution for treatment of emphysema where the entire lung is diseased.

Both bronchodilator drugs and lung reduction surgery fail to capitalize on the increased collateral ventilation taking place in the diseased lung. There remains a need for a medical procedure that can alleviate some of the problems caused by COPD. There is also a need for a medical procedure that alleviates some of the problems caused by COPD irrespective of whether a portion of the lung, or the entire lung is emphysematous.

The present invention addresses the problems caused by COPD by providing a device configured to create collateral openings through an airway wall which allows expired air to pass directly out of the lung tissue responsible for gas exchange. These collateral openings ultimately decompress hyper inflated lungs and/or facilitate an exchange of oxygen into the blood.

Furthermore, there is also a need for devices that are able to access remote areas of the body to provide dual functions of locating an acceptable site for removal or cutting of tissue and then removing or cutting the tissue without having to reposition the device or switch between a separate locator and cutting device. Such a need is evident in dynamically moving environments (e.g., the lungs) where repositioning of a device to find the original target site may be difficult.

Doppler ultrasound is an effective means to determine the presence or absence of a blood vessel within tissue. It is known that sound waves at ultrasonic frequencies travel through tissue and reflect off of objects/interfaces where density gradients exist. In such a case, the reflected signal and the transmitted signal will have the same frequency. Alternatively, in the case where the signal is reflected from the blood cells moving through a blood vessel, the reflected signal will have a shift in frequency from the transmitted signal. This shift is known as a Doppler shift. However, since the characteristics of components used to detect a Doppler shift vary from characteristics of components used to cut or remove tissue, it is difficult to cut or remove tissue in precisely the same location and immediately after detection has taken place. It is usually required that the component or device used to detect any Doppler shift first must be moved to allow a second component or device to cut or remove the tissue at the same precise location. For instance, if a device uses energy to create an opening or ablate tissue, the energy delivery components may not have acceptable characteristics to function as Doppler components. Furthermore, the process of delivering energy through the device may undesirably impact any Doppler components.

When using Doppler in tissue it is noted that the acoustic impedance of the ultrasound transducer and the acoustic impedance of tissue differ significantly. As a result, the ultrasound signal may experience significant reflection and divergence at the tissue/transducer interface. To address this issue, a tip or lens may be used as an interface between the transducer and tissue.

In common Doppler ultrasound applications, a tip material is selected to provide an optimum acoustic match between the ultrasonic transducer and tissue. This optimum acoustic match is the geometric mean impedance between the tissue and the transducer material, governed by the following equation.
Zₒₚₜᵢₘᵤₘ=(Zₜᵢₛₛᵤₑ×Z_{transducer})^{1/2}

Where Zₒₚₜᵢₘᵤₘ is the desired acoustic impedance of the tip material; Zₜᵢₛₛᵤₑ is the acoustic impedance of tissue; and Z_{transducer} is the acoustic impedance of the transducer. Generally, Zₜᵢₛₛᵤₑ ranges from 1.38 MRayls (for fat) to 1.70 MRayls (for muscle), while Z_{transducer} is approximately 30 MRayls for ceramic transducer materials. Therefore, using Z_{transducer} of 1.54 MRayls (the average acoustic impedance for tissue) the desirable tip material should have an acoustic impedance around 6.79 MRayls.

Most materials having an acoustic impedance close to this range are made of epoxy composites and range from, for example, 1.78 MRayls for a methylpentene copolymer (e.g., TPX, Matsui Plastics, White Plains, N.Y.) to 4.39 MRayls for high temperature plastics (e.g., CELAZOLE, Curbell Plastics, Glenshaw, Pa.). Other suitable materials include ceramic (e.g., Macor) which has an acoustic impedance of 14 MRayls.

One drawback to using Doppler ultrasound devices for placing collateral openings in tissue is that conventional tip materials selected for their desirable acoustic impedance are not effective to deliver energy (e.g., RF, resistive heat, etc.) The acoustic impedance of electrically and thermally conductive materials is higher than the desired acoustic impedance of 6.79 MRayls. For example, Zₐₗᵤₘᵢₙᵤₘ is approximately 18 MRayls, Zₜᵢₜₐₙᵢᵤₘ is approximately 27 MRayls, and Z_{stainless steel} is approximately 45 MRayls.

Another drawback to delivering energy through devices configured for Doppler applications is that the transducer is prone to being damaged. For example, when used to deliver therapeutic RF energy, an electrically conductive tip experiences heating. If a sufficient amount of heat is conducted from the tip, the transducer may depolarize. Moreover, conduction of heat through the device may adversely affect the joints and bonds between the transducer, tip and device. As a result, there is the potential of a catastrophic failure of the device if the assembly breaks apart during use in the body.

In view of the above, the present invention provides a device capable of locating an acceptable site for the creation of a collateral opening and then creating an opening in the tissue using a device capable of both functions. While the present invention is discussed as having applicability to creation of collateral openings it was found to have utility for other applications as well. For example, the present invention is suited for the application of energy to tissue in a safe manner (e.g., tumor ablation, tissue removal, application of heat to structures within the body, etc.). Especially when there is a need to avoid blood vessels, or other tissue/organs/structures. The invention has applicability given a need to use of Doppler effect to locate movement within tissue and then apply energy based on the observation of the Doppler effect.

Methods and devices for creating, and maintaining collateral channels are also discussed in U.S. patent application Ser. No. 09/633,651, filed on Aug. 7, 2000; U.S. patent application Ser. Nos. 09/947,144, 09/946,706, and 09/947,126 all filed on Sep. 4, 2001; U.S. Provisional Application No. 60/317,338 filed on Sep. 4, 2001, and 60/334,642 filed on Nov. 29, 2001, and U.S. patent application Ser. Nos. 10/080,344 and 10/079,605 both filed on Feb. 21, 2002.

### SUMMARY OF THE INVENTION

The invention is related to devices for applying energy to tissue. The invention typically involves a catheter having a proximal section, a distal section and a distal end. The catheter comprises an ultrasonic transducer positioned in the distal section. The ultrasonic transducer is adapted to emit and receive ultrasonic signals. The ultrasonic signals may be analyzed to determine whether a blood vessel is present in the vicinity of the catheter's distal end. The ultrasonic transducer may be a piezoelectric transducer.

The catheter also includes a tip assembly located at and forming the distal end of the catheter. The tip assembly comprises an acoustically-transmitting material such as ceramic and an electrode at least partially coating (or otherwise attached to) the acoustically-transmitting material. The electrode may be a metal ring, thin coating, a ring-shaped coating, or any of a number of shapes and coatings.

In one variation of the present invention, the acoustically-transmitting material is electrically nonconducting, electrically separating the electrode from the ultrasonic transducer. Also, the electrode and the ultrasonic transducer are positioned such that when ultrasonic signals are emitted and received by the ultrasonic transducer the signals are transmitted through the distal end of the catheter and in some variations, through at least a portion of the electrode. The electrode may be titanium, stainless steel, or a number of other types of electrically conducting materials.

The acoustically-transmitting material may be positioned adjacent to a distal surface of the ultrasonic transducer. Also, the acoustically-transmitting material may be adhered to the distal surface of the ultrasonic transducer. The acoustically-transmitting material may have an axial length that is optimized for acoustic energy transmission. For example, the axial length may be up to 0.9 mm and in some variations, it may range from 0.04 to 0.86 mm. The acoustically transmitting material is ceramic in one variation of the present invention.

A flexible tubular member may coaxially surround the ultrasonic transducer. The tubular member may extend partially over the acoustically-transmitting material such that a distal edge of the tubular member mates with a proximal edge of the electrode coating. The flexible tubular member may be polyimide.

In another variation of the invention, the tip assembly can have a flat end. Also, the distal section of the catheter may have a constant outer diameter.

In another variation of the invention, a metal conductive member is placed in electrical contact with a proximal surface of the ultrasonic transducer element and a first conductive wire is placed in electrical contact with the metal conductive member. The metal conductive member may be a metal tube such as a hypotube. Also, a backing layer may be disposed within the metal tube. The backing layer is made of material which does not acoustically transmit ultrasonic waves.

The ultrasonic transducer may comprise a conductive coating disposed over at least a portion of a distal surface of an ultrasonic transducer element. A second conductive wire is placed in electrical contact with the conductive coating disposed over at least a portion of the distal surface of the ultrasonic transducer element.

Also, a third conducting element may be connected to the electrode and the third conducting element is electrically insulated from the ultrasonic transducer. The third conducting wire may supply RF signals to the electrode.

In another variation an acoustic transducer assembly comprises a piezoelectric material comprising a first surface and a second surface opposite the first surface; a first metallic member in contact with the first surface of the piezoelectric material; a second metallic film at least partially coating the second surface of the piezoelectric material; and an acoustically transmitting material having a proximal surface and a distal surface wherein the proximal surface of the acoustically transmitting material is fused to the second metallic film with heat.

In another variation of the invention, an electrically conducting material may be disposed on at least a portion of the distal surface of the acoustically transmitting material. The electrically conducting material may be in the shape of a ring. The electrically conducting coating may partially or completely cover the distal surface of the acoustically transmitting material.

The acoustic transducer assembly may have various shapes including, for example, a cylindrical shape. Also, a polymeric sleeve may at least partially coaxially surround the assembly.

### BRIEF DESCRIPTION OF THE DRAWINGS

FIGS. 1A-1C illustrate various states of the natural airways and the blood-gas interface.

FIG. 1D illustrates a schematic of a lung demonstrating a principle of the effect of collateral channels placed therein.

FIGS. 2A-2C are side views of devices having an electrically conductive tip which is able to function as a Doppler tip as well as create the collateral channels.

FIG. 2D illustrates an insulating layer on a device.

FIGS. 3A-3H illustrate examples of tip configurations.

FIGS. 4A-4B illustrate cross sectional views of examples of transducer assemblies.

FIGS. 5A-5E illustrate various configurations used to deliver energy to the tip of a device.

FIGS. 6A-6B show sectional views of a device where a conductive medium also serves to attach a tip to the device.

FIGS. 7A-7E illustrate various configurations to retain a tip to devices.

FIGS. 8A and 8B are a perspective view and a cross sectional view respectively of a multifunctional tip configuration.

FIGS. 8C and 8D are a perspective view and a cross sectional view respectively of another multifunctional tip configuration.

FIGS. 8E and 8F are a perspective view and a cross sectional view respectively of an ultrasonic transducer.

FIGS. 8G and 8H are a perspective view and a cross sectional view respectively of another ultrasonic transducer assembly.

FIGS. 8I and 8J are a perspective view and a cross sectional view respectively of yet another ultrasonic transducer assembly.

FIGS. 9A-9C depict a device creating a collateral channel in the airways of the lung.

FIGS. 10A-10B illustrate time-of-flight diagrams for the Doppler echo signal used to determine the Doppler control settings.

FIG. 10C illustrates an example of a schematic representation of a pulsed wave Doppler electronic system for use with the inventive device.

### DETAILED DESCRIPTION OF THE INVENTION

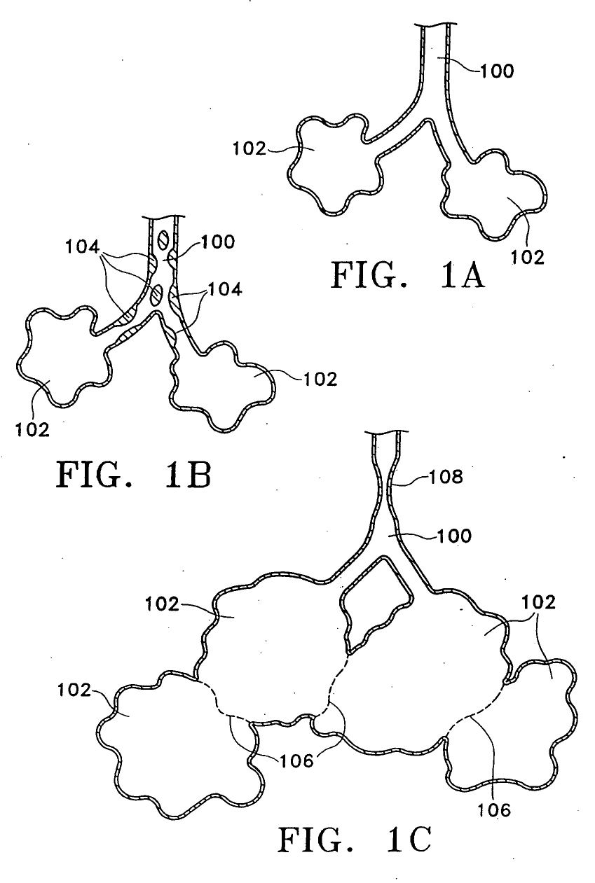

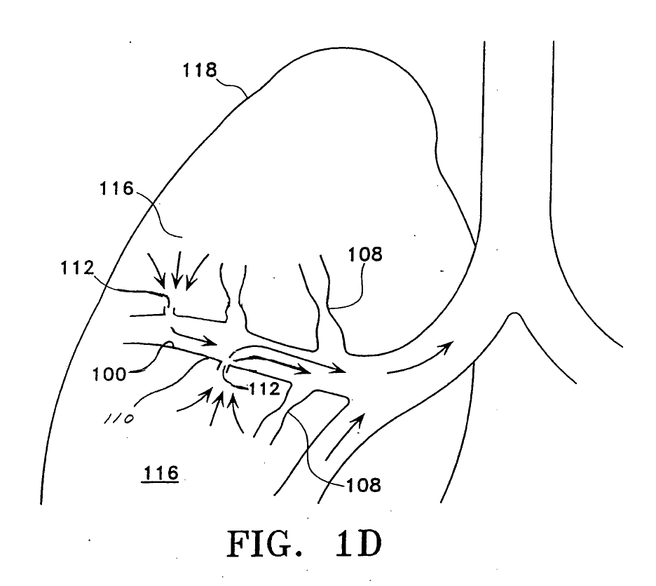

Prior to considering the invention, simplified illustrations of various states of a natural airway and a blood gas interface found at a distal end of those airways are provided in FIGS. 1A-1C. FIG. 1A shows a natural airway 100 which eventually branches to a blood gas interface 102. FIG. 1B illustrates an airway 100 and blood gas interface 102 in an individual having COPD. The obstructions 104 (e.g., excessive mucus resulting from COPD, see above) impair the passage of gas between the airways 100 and the interface 102. FIG. 1C illustrates a portion of an emphysematous lung where the blood gas interface 102 expands due to the loss of the interface walls 106 which have deteriorated due to a bio-chemical breakdown of the walls 106. Also depicted is a constriction 108 of the airway 100. A combination of the phenomena depicted in FIGS. 1A-1C are often found in the same lung.

The following text and corresponding figures provide variations and embodiments of the present invention. It is contemplated that combinations of features of the specific embodiments or combinations of the specific embodiments themselves are within the scope of the present invention.

The production and maintenance of collateral openings or channels through airway walls permits air to pass directly out of the lung tissue and into the airways to ultimately facilitate exchange of oxygen into the blood and/or decompress hyper inflated lungs. The term ‘lung tissue’ is intended to include the tissue involved with gas exchange, including but not limited to, gas exchange membranes, alveolar walls, parenchyma and/or other such tissue. To accomplish the exchange of oxygen, the collateral channels allow fluid communication between an airway and lung tissue. Therefore, gaseous flow is improved within the lung by altering or redirecting the gaseous flow within the lung, or entirely within the lung.

FIG. 1D illustrates a schematic of a lung 118 to demonstrate a benefit of the production and maintenance of collateral openings or channels through airway walls. As shown, a collateral channel 112 (located in an airway wall 110) places lung tissue 116 in fluid communication with airways 100 allowing air to directly pass out of the airways 100.

The term ‘channel’ is intended to include, without limitation, any opening, cut, hole, slit, tear, puncture, or any other conceivable artificially created opening. The channel may be created in tissue having a discrete wall thickness and the channel may extend all the way through the wall. Also, a channel may extend through lung tissue which does not have well defined boundaries such as, for example, parenchymal tissue.

As shown, constricted airways 108 may ordinarily prevent air from exiting the lung tissue 116. In the example illustrated in FIG. 1D, there is no implanted structure placed in the collateral channel 112. However, conduits (not shown) may be placed in the collateral channels 112 to assist in maintaining the patency of the collateral channels 112. Examples of conduits may be found in the applications discussed above. While there is no limit to the number of collateral channels which may be created, it is preferable that 1 or 2 channels are placed per lobe of the lung. For example, the preferred number of channels is 2-12 channels per individual patient. In current trials, it was found that 1-4 channels placed per lobe of the lung and 4-16 channels per individual patient was preferable. This number may vary on a case by case basis. For instance, in some cases an emphysematous lung may require 3 or more collateral channels in one or more lobes of the lung.

In the following explanation of figures, similar numerals may represent similar features for different variations of the invention.

The devices of the present invention are configured to locate a target site for creation of a collateral channel in the tissue and to create an opening in tissue. As discussed above, a benefit of providing a single device having both capabilities is that the device can be used to select a target location and then create an opening without needing to be moved. That is, two separate devices do not need to be switched out to accomplish the selecting and channel-creating steps. Although the device is discussed as being primarily used to create channels in the lungs, the device is not limited as such and it is contemplated that the invention has utility in other areas as well, specifically in applications in which blood vessels or other structures must be avoided while cutting or removing tissue (one such example is tumor removal.)

A device disclosed herein is able to detect the presence or absence of a blood vessel by placing a front portion of the device in contact with tissue. Doppler ultrasound may be used to detect the presence of blood vessels within tissue. However, the frequency of the signals is not limited to the ultrasonic range, for example the frequency may be within the range of human hearing, etc. Other sources of energy may be used to detect the presence or absence of a structure. The other forms of energy include, for example, light or heat.

The ultrasound Doppler operates at any frequency in the ultrasound range but preferably between 2 Mhz-30 Mhz. It is generally known that higher frequencies provide better resolution while lower frequencies offer better penetration of tissue. In the present invention, because location of blood vessels does not require actual imaging, there may be a balance obtained between the need for resolution and for penetration of tissue. Accordingly, an intermediate frequency may be used (e.g., around 8 Mhz). A variation of the invention may include inserting a fluid into the airway to provide a medium for the Doppler sensors to couple to the wall of the airway to detect blood vessels. In those cases where fluid is not inserted, the device may use mucus found within the airway to directly couple the sensor to the wall of the airway.

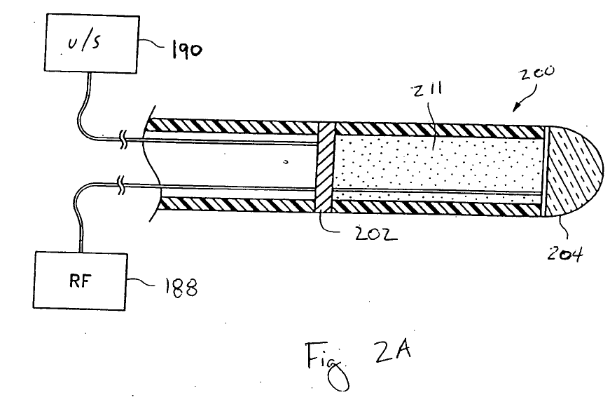

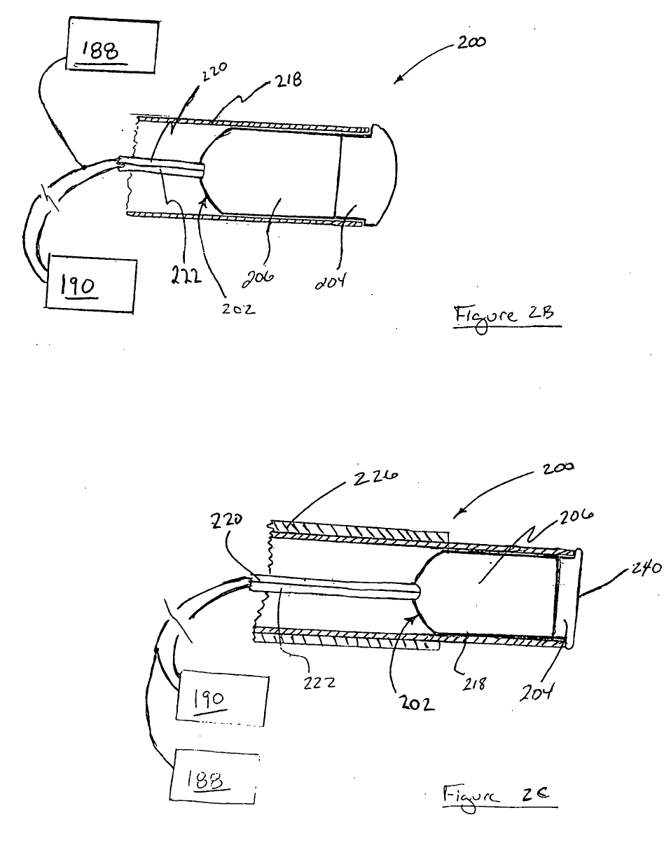

FIG. 2A illustrates a variation of a device 200 where a tip 204 of the device has a conductive portion, (e.g., it is made from a conductive material or has a conductive coating) allowing the tip to serve as both an acoustic energy transmitter (or lens) and an RF electrode. Accordingly, the tip 204 is connected to an RF generator 188 for creating channels within tissue and a transducer assembly 202 is placed in communication with an analyzing device 190 that is adapted to measure the Doppler shift between generated and reflected signals. It is contemplated that, throughout this disclosure, the transducer assembly 202 may be a transducer or a transducer coupled with a covering and other components. In this variation, the tip 204 may be separated from the transducer 202, but both the tip 204 and transducer 202 are in acoustic communication through the use of a separation medium 211. The separation medium 211 transmits signals between the tip 204 and the transducer 202. In some variations of the invention, the spacing of the transducer 202 from the tip 204 serves to prevent heat or RF energy from damaging the transducer 202. It is intended that the spacing between the transducer 202 and tip 204 shown in the figures is for illustration purposes only. Accordingly, the spacing may vary as needed. The separation medium must have acceptable ultrasound transmission properties and may also serve to provide additional thermal insulation as well. For example, an epoxy as described herein, may be used for the separation medium.

The separation medium may provide electrical separation between the ultrasonic transducer and the tip 204. For example, the tip 204 may be separated from the ultrasonic transducer with a ceramic material. Other materials that may serve to separate the tip from the ultrasonic transducer include glass, aerogel and other materials that have low acoustic impedance; that are electrically insulating; and/or that are thermally insulating. Use of these materials can reduce noise in the ultrasonic signal, increasing sensitivity.

It is also contemplated that the inventive device may create openings in tissue using any type of energy capable of removing/ablating tissue. For example, RF energy or focused ultrasound may be used.

FIG. 2B illustrates a sectional side view of a variation of the inventive device 200. The device 200 includes a transducer assembly 202. As shown in the figure, an electrically conductive tip 204 is adjacent to the transducer assembly 202 and at a distal end of the elongate member 218. The transducer assembly 202 is located towards a distal portion of the elongate member 218. The transducer assembly of any variation of the present invention may be located within the elongate member, or it may be located within a portion of the tip of the device. In any case, the transducer assembly will be located towards the distal portion of the elongate member. The elongate member 218 of the present invention may or may not have a lumen extending therethrough. The elongate member described herein may be comprised of any commercially available medical-grade flexible tubing. Furthermore, the elongate member may be selected from material that provides insulation from the heat generated by the device. For example, the elongate member may comprise a PTFE material. In such cases, the elongate member will provide insulation for tissue that is adjacent to the area where creation of a collateral channel is desired. Also, in some cases, insulation may be required to prevent damage to the transducer assembly.

The device 200 further includes a first conducting member 220 and a second conducting member 222 (e.g., wires) both extending through at least a portion of elongate member 218 to the transducer assembly 202. The conducting members 220, 222 may extend through a lumen of the elongate member 218 or may extend in the wall of the elongate member 218. In any case, the conducting members 220, 220 provide the energy and controls for the transducer assembly 202. For example, the conducting members 220, 222 may be coupled to an ultrasound source 190. Moreover, variations of the inventive device include conducting members 220, 222 which may be comprised of a series of wires, with one set of wires being coupled to respective poles of the transducer, and any number of additional sets of wires extending through the device. Ultimately, the wires enable the device to couple to energy and control units. Although not illustrated, the device 200 may also include an outer sheath (not shown in FIG. 2B) in which the device 200 may be advanced to a target tissue site.

FIG. 2C illustrates another variation of a device 200 for creating collateral channels. In this variation, a transducer assembly 202 is provided with a conductive tip 204 having a flatter front surface 240. It should be noted that the shape of the tips illustrated in FIGS. 2A-2C are intended to illustrate examples of tips for the present invention, the shapes of the tips are not meant to be limited to any particular variation of the device. The tip 204 is located adjacent to a covering 206 of the transducer assembly 202. The transducer assembly 202 is located towards a distal portion of the elongate member 218. In the variation depicted in FIG. 2C the device 200 also includes an (optional) outer sheath 226. As illustrated, the conductive tip 204 may be coupled to an energy source 188 using one of the conducting members 220 or 222. In such a case, the tip 204 will be electrically coupled to one of the conducting members.

Although the transducer assembly is adapted to generate a source signal and receive a reflected signal, variations of the invention may omit the transducer covering and other structures not necessary to generate a source signal and receive a reflected signal. Therefore, it is contemplated that the invention may simply have a transducer that is coupled to a controller.

FIG. 2D illustrates a variation of the device 200 with an insulating layer 264 on the distal end of the device 200. The insulating layer 264 may be a coating, sleeve, etc. which prevents heat generated by the device from adversely affecting either tissue or the transducer assembly. The insulating layer 264 may extend over a limited area of the device as needed. Examples of the insulating layer 264 materials include polyimide, silicone, PTFE, FEP, and PFA.

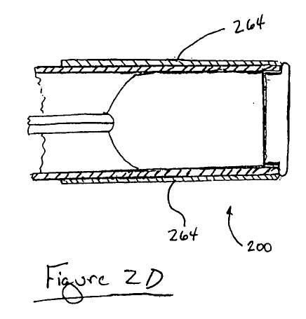

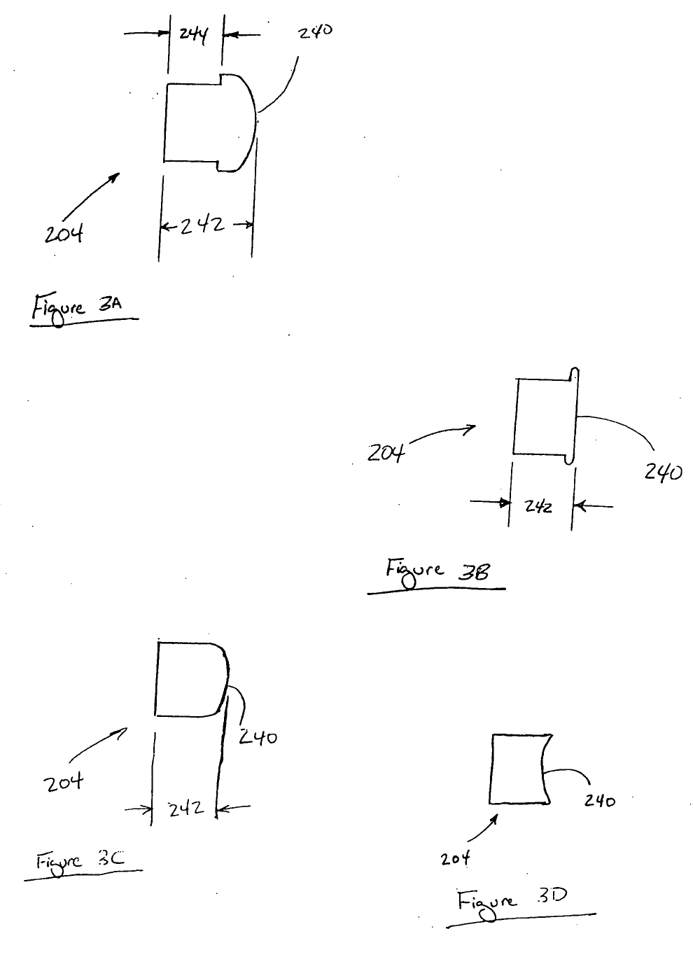

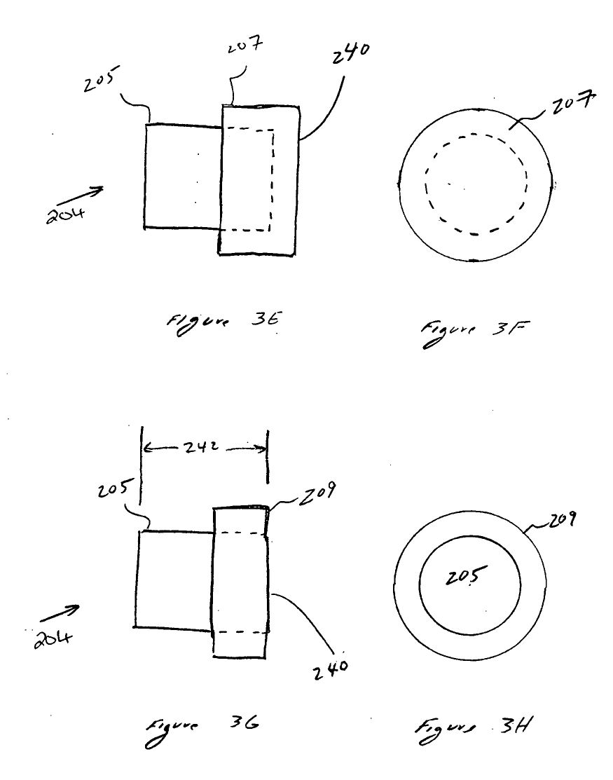

FIGS. 3A-3D illustrate possible variations of the tip 204 of the device. It is noted that these variations are provided for illustrative purposes and are not meant to be limiting. The tips 204 of the present invention may function as a lens to disperse and/or direct a signal over a substantial portion of the outer surface of the tip 204. The tip 204 also may be adapted to disperse and/or direct (e.g., by diffraction) a reflected signal towards the transducer (not shown in FIGS. 3A-3D). Accordingly, given the above-described configuration, the inventive device 200 will be able to detect vessels with substantially most of the tip 204. Because most of the tip 204 is able to direct a signal to and from the transducer 208, this device 200 may detect vessels through a greater range of contact angles (e.g., as opposed to requiring the device 200 to be orthogonal to the tissue.) Furthermore, the tip may comprise a directing means such as a prism or one more apertures having varying sizes, shapes and locations.

The tip 204 may be designed such that it interferes and redirects the signals in a desired direction in a manner like a lens. It also may be desirable to place an epoxy between the tip 204 and the transducer. Preferably, the epoxy is thin and applied without air gaps, bubbles or pockets. Also, the density/hardness of the epoxy should provide for transmission of the signal while minimizing any effect or change to the source signal. The configuration of the transducer assembly 202 permits the tip 204 to disperse a signal over a substantial portion of its outer surface 240. The tip 204 also is adapted to refract a reflected signal towards the transducer 208. Accordingly, given the above-described configuration, the inventive device will be able to detect vessels with any part or substantially all of the lens 204 that contacts tissue.

Although the tip is able to transmit a source signal and receive a reflected signal, the device is not limited to requiring both functions. For example, a device could be configured to generate a source signal and direct the source signal to an area of interest and a second device or transducer assembly could be used to receive the reflected signal. Accordingly, one device could be used to generate the source signal and a separate device may be used to receive the reflected signal.

The tip 204 may also comprise an electrically or thermally conductive material such that energy (e.g., RF energy or thermal energy) may be delivered to the tissue via the tip 204. For example, the tip may be comprised of titanium, aluminum, stainless steel, etc., or any electrically or thermally conductive metal. Also, the tip 204 may be comprised of any material suitable for ultrasound applications but is not particularly electrically conductive. In such a case, the tip will have an electrically conductive coating about at least a portion of the tip. These tip materials include dimethyl pentene, a methylpentene copolymer (plastic-TPX), carbon aerogel, polycarbonate (e.g., Lexan), polystyrene, Macor ceramics, glass, various epoxies, etc. (e.g., any standard material used for ultrasound applications.) Electrically conductive coatings include gold, silver, tantalum, copper, chrome, aluminum, stainless steel, platinum, titanium, or any biocompatible electrically conductive material, etc. This material may be coated, deposited, plated, painted, wound, wrapped (e.g., a conductive foil), etc. onto the tip 204.

As discussed above, traditional tip materials are selected to provide an optimum acoustic match between the ultrasonic transducer and tissue. Use of such electrically conductive materials do not provide optimum acoustic impedance in Doppler applications. To overcome the problem associated with tip materials having undesirable acoustic impedance, the tip 204 of the present invention is selected to be long enough to avoid excessive heating of the transducer 208 and at a length that minimizes the signal clutter resulting from the use of material.

In view of the above, a tip 204 length is selected in accordance with the following equation:
L=N(λ/4) for Ztransducer>Ztip>Ztissue

Where L=tip length; N=any integer; and λ=wavelength of the signal travelling in the desired medium. It was found that the best performance was obtained by selecting a tip length where N is an odd integer. This minimizes the destructive interference of the signal caused by out of phase reflections of the signal at the boundaries of the tip. It was also found that while N=1 was acceptable for the Doppler function, the resulting tip length caused undesirable heating of the transducer. To achieve a balance of a tip length that would prevent unacceptable heating of the transducer, N was chosen to be 7 for one variation of the device. Accordingly, an acceptable length for a titanium tip corresponding to a frequency of 8 Mhz, equals 1.33 mm or 0.052 in.

A measurement of the tip lengths 242 may be seen in FIGS. 3A-3D. FIG. 3A illustrates a variation of the tip 204 having a rounded front surface 240. In this case, the tip length 242 of the entire tip may be selected such that N is an odd integer (e.g., 9) and the length behind the front surface 244 may be selected to be any integer multiple of the wavelength (e.g., 6 or 7). In such an example the length of 242 may be selected, for example, L₂₄₂=9(λ/4) and L₂₄₄=7(λ/4).

As illustrated in FIG. 3A, although the front surface 240 of the tip 204 is illustrated as being hemispherical, the tip 204 may have other profiles as well. For example, it is desirable that the tip 204 produce a certain amount of divergence of the signal being passed therethrough. However, depending on a variety of factors (e.g., material, frequency of the signal, etc.) a tip 204 may encounter excessive divergence which is destructive to the outgoing signal. Accordingly, it may be desirable to produce a tip 204 as illustrated in FIG. 3B in which a front surface 240 of the tip 204 is substantially flat. The degree of flatness of the tip 204 will often depend upon experimentation to reduce the amount of destructive reflections, thus minimizing excessive divergence due to differences in speed of sound in tip versus tissue. Use of materials with higher acoustical impedance, such as titanium and stainless steel, may require a flatter tip due to the resulting divergence of the source signal. FIG. 3C illustrates another variation of a tip 204 having a rounded front surface 240 but with no projections on the sides of the tip 204. FIG. 3D illustrates a tip 204 with a concave front surface 240.

FIGS. 3E and 3F are side and front views respectively of another variation of a tip 204 having a flat surface 240. In particular, tip 204 has a substrate material 205 partially coated with a conductive material 207. The substrate material may be selected to transmit ultrasonic energy as well as isolate (electrically and/or thermally) the tip from the transducer (not shown). The substrate material 205 may also transmit ultrasonic energy. Substrate materials include, without limitation, polymers as described above as well as ceramics. The conductive material 207 may be a metallic coating (e.g., titanium) or another conductive material (e.g., a stainless steel) attached to the substrate material.

FIGS. 3G and 3H are side and front views respectively of another variation of a tip 204 having a flat surface 240. In particular, tip 204 has a substrate material 205, a portion of which is coaxially surrounded with a conducting material 209. The conducting material may be a material sputtered or otherwise disposed on the substrate or it may be a material mounted or attached to the substrate. For example, a stainless steel ring may be press fit or glued to the substrate. Other materials than those specified above may be employed in the present invention.

The length L 242 of the tip may be defined by the equation set forth above. That is, L=N×( [symbol] /4) where N is an odd integer and [symbol] is the wavelength of the ultrasonic wave traveling through the material. If, for example, the substrate material 205 is aluminum oxide (velocity of sound traveling in aluminum oxide is 11,000 meters per second) and the ultrasound wave is activated at frequency of 8×10⁶ cycles per second, the corresponding wavelength [symbol] is about 0.14 mm per cycle. The length 242 thus may range from 0.04 mm (N=1) to 0.28 mm (N=7) and perhaps, from 0.04 mm to less than 0.9 mm for greater values of N. The above example is for illustrative purposes and is not intended to be limiting. Also, the ultrasound frequency may vary from 8×10⁶. For example, the ultrasound frequency may range from 2 to 30 MHz or perhaps, 6 to 10 MHz. Also, as indicated above, the choice of material chosen affects this calculation and must be compensated for when selecting a length.

It may also be desirable that the device is configured such that there are no exposed sharp edges that may cause any unintended damage to tissue while the device is being used to determine the presence or absence of a blood vessel. In such a case, for example, the tip may be designed such that it doesn't have sharp edges, or any sharp edges may be covered by other parts of the device (e.g., the elongate member, an outer sheath, etc.)

As discussed herein, for some variations of the invention it is desirable to minimize the size of the device especially at the distal end. Although the invention may be any size, it was found that an overall device diameter of 0.071″ was acceptable. As noted, because the device is advanced through the airways, the device may treat deeper areas in the airways of the lungs given a smaller outside diameter of the distal end of the device. This size also permits delivery of the device into the lungs through the working channel of a standard bronchoscope or endoscope. However, this reduction in size is limited as functionality of the device may suffer. For example, one or more wires will be selected such that they will deliver sufficient RF energy over a desired period of time without experiencing unacceptable heating. Therefore, the smallest acceptable cross sectional area of a single wire or multiple wires will be a balance of the energy delivery requirements of the device versus the characteristics of the wire or wires.

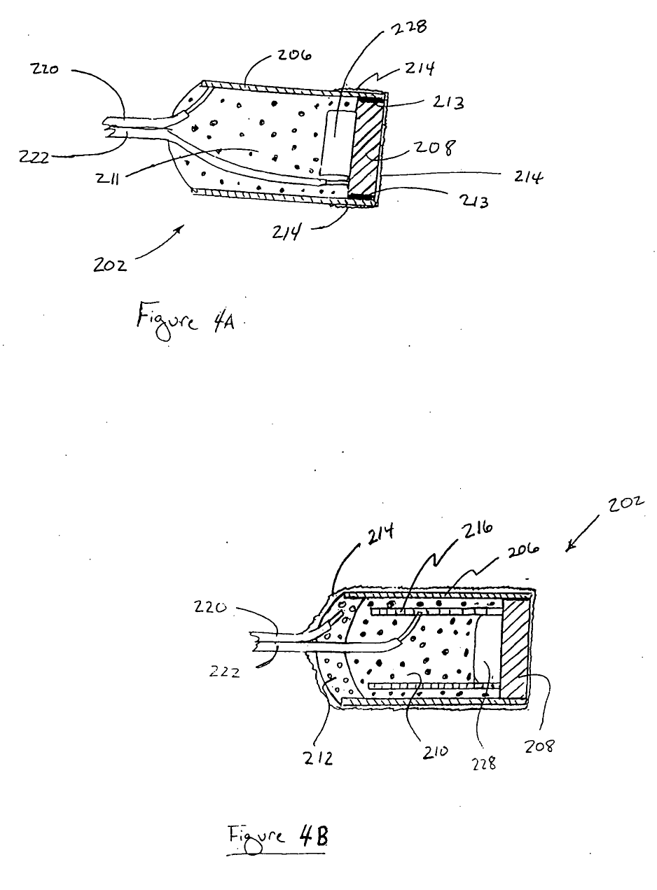

FIGS. 4A-4B illustrate variations of the transducer assembly 202 which are configured to reduce an overall size of the assembly. FIG. 4A illustrates a cross-sectional view of a basic variation of a transducer assembly 202. For illustration purposes, the transducer assembly 202 illustrated in FIG. 4A is shown without a tip. The transducer assembly 202 includes at least one transducer 208 (e.g., a piezoelectric transducer.) In this variation, the front surface of the transducer 208 comprises a first pole and the rear surface comprises a second pole.

The transducer or transducers may comprise a piezo-ceramic crystal (e.g., a Motorola PZT 3203 HD ceramic). A single-crystal piezo (SCP) may be used as well as other types of materials including, without limitation, ferroelectric materials such as poly-crystalline ceramic piezos, polymer piezos, or polymer composites. The substrate may typically be made from piezoelectric single crystals (SCP) or ceramics such as PZT, PLZT, PMN, PMN-PT; also, the crystal may be a multi layer composite of a ceramic piezoelectric material. Piezoelectric polymers such as PVDF may also be used. Micromachined transducers, such as those constructed on the surface of a silicon wafer are also contemplated (e.g., such as those provided by Sensant of San Leandro, Calif.) As described herein, the transducer or transducers used may be ceramic pieces coated with a conductive coating, such as gold. Other conductive coatings include sputtered metal, metals, or alloys, such as a member of the Platinum Group of the Periodic Table (Ru, Rh, Pd, Re, Os, Ir, and Pt) or gold. Titanium (Ti) is also especially suitable. The transducer may be further coated with a biocompatible layer such as Parylene or Parylene C.

The covering 206 of the transducer assembly 202 contains the transducer 208. In some variations of the invention, the covering 206 may comprise a conductive material. In such cases the covering 206 itself becomes part of the electrical path to the first pole of the transducer 208. Use of a conductive covering 206 may require insulating material 213 between the sides of the transducer 208, thereby permitting a first conductive medium 214 to electrically couple only one pole of the transducer 208 to the covering 206.

At least a portion of the front surface of the transducer 208, will be in contact with the conductive medium 214. The conductive medium 214 permits one of the poles of the transducer 208 to be placed in communication with a conducting member that is ultimately coupled to a power supply. As shown in this example, the conductive medium 214 places the pole of the transducer 208 in electrical communication with the covering 206. In some variations the conductive medium 214 may coat the entire transducer 208 and covering 206. Alternatively, the conductive medium 214 may be placed over an area small enough to allow for an electrical path between a conducting member and the respective pole of the transducer 208. The conductive medium 214 may be any conductive material (e.g., gold, silver, tantalum, copper, chrome, or any bio-compatible conductive material, etc. The material may be coated, deposited, plated, painted, wound, wrapped (e.g., a conductive foil), etc. onto the transducer assembly 202.

The transducer assembly 202 depicted in FIG. 4A also illustrates conducting members 220, 222 electrically coupled to respective poles of the transducer 208. Optionally, the conducting members 220, 222 may be encapsulated within an epoxy 211 located within the covering 206. The epoxy 211 may extend to the transducer 208 thereby assisting in retaining both the conducting members 220, 222 and transducer 208 within the covering. It may also be desirable to maintain a gap 228 between the transducer 208 and any other structure.

FIG. 4B illustrates another variation of a transducer assembly 202. In this variation, the conductive medium 214 extends over the entire transducer covering 206. Accordingly, the covering 206 may be made of a non-conducting material (e.g., a polyamide tube, polyetherimide, polycarbonate, etc.) The transducer assembly 202 may further comprise a second tube 216 within the covering 206. This second tube 216 may be a hypo-tube and may optionally be used to electrically couple one of the conducting members to a pole of the transducer 208. As shown, the covering 206 may contain a non-conductive epoxy 210 (e.g., Hysol 2039/3561 with Scotchlite glass microspheres B23/500) which secures both the conducting member and the second tube 216 within the covering 206. This construction may have the further effect of structurally securing the transducer 208 within the assembly 202. Again, a gap 228 may or may not be adjacent to the transducer to permit displacement of the transducer 208.

FIG. 4B also illustrates the assembly 202 as having a conductive epoxy 212 which encapsulates the alternate conducting member 220. An example of a conductive epoxy is Bisphenol epoxy resin with silver particulates to enable conductivity. The particulates may be from 70-90% of the resin composition. The resin may then be combined with a hardener (e.g., 100 parts resin per 6 parts hardener.) The conductive epoxy 212 is in electrical communication with the conductive medium 214 allowing for a conductive path from the conducting member 220 to the conductive medium 214. Accordingly, use of the conductive epoxy 212 secures the conducting member 220 to the assembly 202 while electrically coupling the conducting member 220 to the transducer via the conductive coating 214.

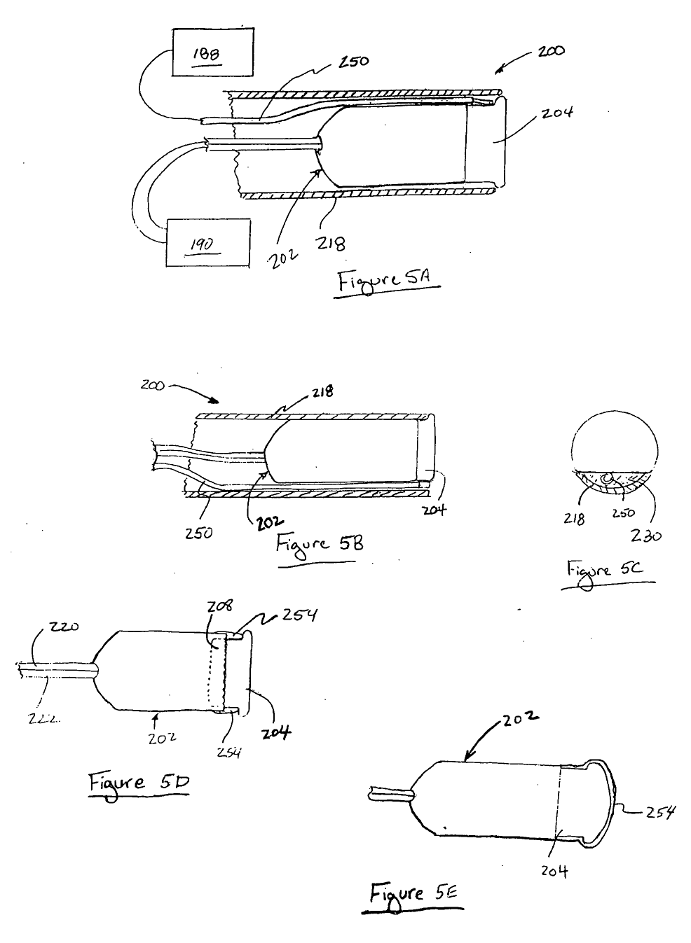

FIG. 5A illustrates a variation of the inventive device 200 having a conductive tip 204 located at the front of the transducer assembly 202. As illustrated, the conductive tip 204 may have a third conducting member (e.g., a wire) electrically coupled directly to the conductive tip 204. However, this configuration requires an elongate member 218 with a diameter larger than that of the transducer assembly 202 to accommodate a wire along side of the transducer assembly 202. It may be desirable to minimize the diameter of the transducer assembly 202 so that the device 200 may fit within the working channel of a bronchoscope or other endoscope. FIG. 5B illustrates another variation of the inventive device 200 which attempts to minimize the size of the elongate sheath 218. As illustrated in FIG. 5B, the transducer assembly 202 may have an outer perimeter that is smaller than an inner perimeter of a lumen of the elongate member 218 such that the third conducting member 250 extends along the lumen and parallel to the transducer assembly 202. As shown in FIG. 5C, which is a side view of the variation of FIG. 5B, this variation of the transducer assembly 202 has a non-circular shape to permit passage of the third conducting member 250 along the side of the transducer assembly 202. As shown, the elongate member 218 may have a retaining epoxy 230 placed within the elongate member 218 to secure the third conducting member 250 and to seal any opening in the distal end caused by the difference in size between the transducer assembly 202 and the elongate member 218.

FIG. 5D illustrates another variation used to minimize the size of the device. For sake of illustration, FIG. 5D only illustrates the transducer assembly 202, conducting members 220, 222, tip 204, and transducer 208 (hidden lines.) As discussed above, the transducer assembly 202 will have a conductive medium (not shown) placed on an outside surface.

FIG. 5D illustrates a second conductive medium 254 placing the tip 204 in electrical communication with the first conductive medium (not shown.) This configuration permits delivery of energy to the tip 204 via one of the conducting members 220 or 222. Therefore, the need for a separate conducting member is eliminated. It should be noted that the amount of second conductive medium 254 is shown for illustrative purposes only. Moreover, the second conductive medium 254 may be located between the tip 204 and the transducer 208. In such a case, an epoxy (not shown) may be used to secure the tip 204 to the transducer assembly 208. The second conductive medium 254 may be any conductive material (e.g., gold, silver, tantalum, copper, chrome, or any bio-compatible conductive material, etc.) Furthermore, the second conductive material 254 may be different or the same material as the first conductive material. In the latter case, the device will appear to have a single conductive material. In FIG. 5D, the second conductive medium 254 is shown to be a coating or deposition. However, as discussed herein, the conductive mediums are not limited as such.

When using a second conductive medium 254 to provide the energy supply to a conductive tip 204 it may be desirable to provide a conductive medium 254 of sufficient thickness so that the energy delivered to the tip 204 does not produce unwanted heating of the overall transducer assembly 202. As discussed above, conducting member(s) were sized to provide sufficient energy while minimizing heating of the member. In practice, the device used gold foils having a thickness ranging from 2-10 microns. However, the conducting members' size and shape may vary from the examples provided herein.

FIG. 5E illustrates a variation of the device where the tip 204 of the transducer assembly 202 is covered with the second conductive material 254. Such a configuration may be used when using a tip 204 that is not bio-compatible. For example, as discussed above, a tip 204 comprised of aluminum may offer excellent acoustic characteristics. However, an aluminum tip 204 may not offer the desired bio-compatibility. Accordingly, coating the tip 204 with the second conductive material 254 where it is exposed to tissue may provide the desired bio-compatibility characteristics. In this configuration it will be necessary to provide the second conductive material 254 in sufficient amounts such that it may deliver sufficient energy to the tip 204 while not reducing performance of the transducer assembly 202. It was found that in using an aluminum tip 204 a gold coating of 5-10 microns was sufficient to deliver sufficient energy to the tip 204. Moreover, because 10 microns corresponds to approximately 1/40th of a wavelength (when using 8 Mhz frequency), the thickness of the coating provided very little signal degradation.

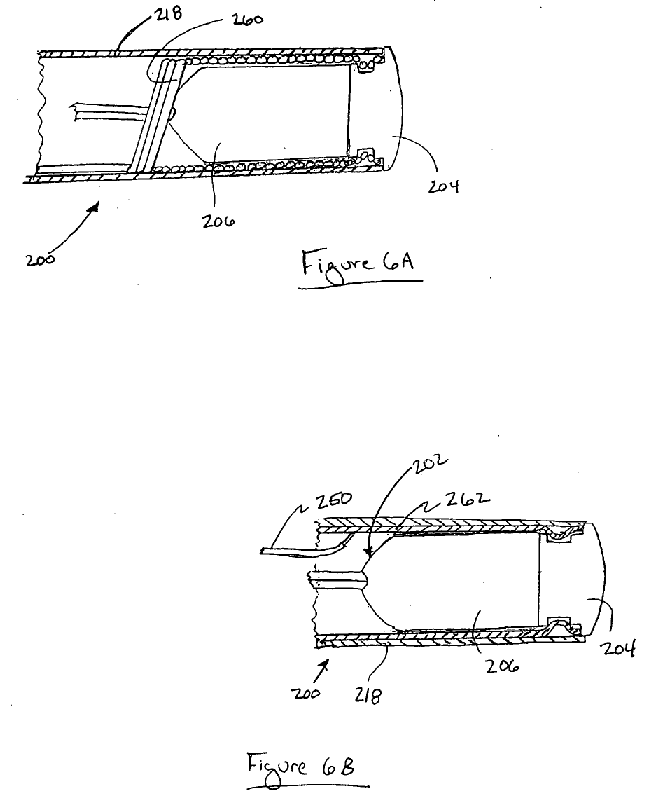

FIG. 6A illustrates a variation of the inventive device where the second conductive medium is formed from a spring 260. The spring 260 may be formed from one or more spring wound wires. The wire(s) forming the spring 260 may extend through the device but ultimately couple to an energy source. This figure also shows insulation between the spring 260 and the covering 206. The insulation may be disposed between any exposed portion of any conducting members. For example, it was found that two wires of 0.005″ diameter wound into a spring was of sufficient size to conduct sufficient current to the tip 204 without resulting in unwanted heating of the wires. Or, the spring 260 may be coupled to the covering 206 or one of the conducting members for delivery of the energy through the spring 260 to the tip 204. As illustrated, the spring 260 may optionally be secured (e.g., crimped, welded, soldered, glued, or reduced in diameter) about the tip 204 to further retain the tip 204. Moreover, a beneficial feature of the spring 260 is that it provides additional flexibility to the end of the device when articulated in a bronchoscope.

FIG. 6B illustrates another variation of the inventive device 200. In this variation, the second conductive medium comprises a tube 262. The tube 262 may be independently connected to an energy source via a third conducting member 250 (as illustrated.) In such cases, it may be necessary to insulate respective portions of the tube 262 from parts of the transducer assembly 202. Alternatively, the tube 262 may be in electrical communication with a portion of the transducer assembly 202 which supplies the energy to the tip 204. As shown, the tube 262 may optionally be secured (e.g., crimped, or reduced in diameter) about the tip 204. It is noted that the tube 262 may have a cross-sectional shape to match the outer shape of the transducer assembly 202 (e.g., circular, oval, rectangular, etc.) The tube 262 may be a hypo-tube comprised of any conductive and preferably bio-compatible material.

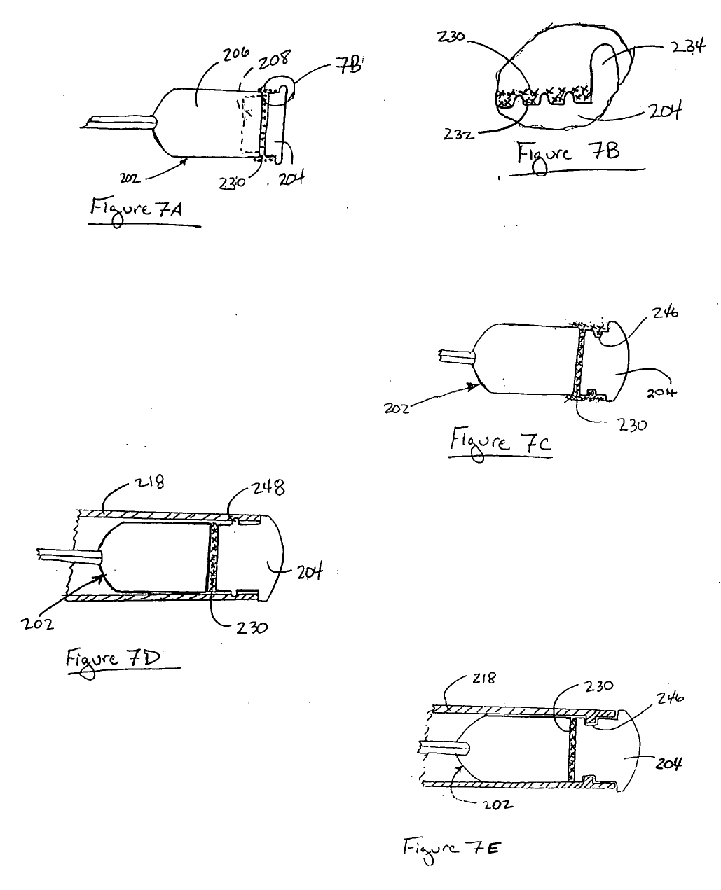

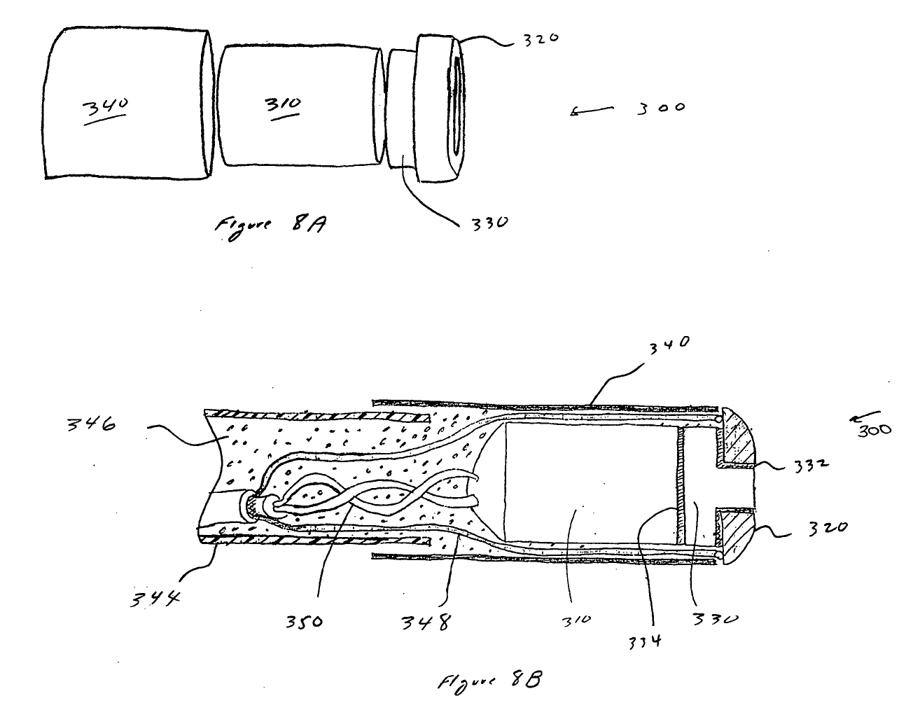

FIGS. 7A-7E illustrate examples of configurations for redundant joints to retain the tip 204 with the device by increasing the retention strength of the tip 204 within the device. It is contemplated that these concepts may be combined as necessary with the variations of the invention disclosed herein.

FIG. 7A illustrates a tip 204 attached to the transducer assembly 202. The tip 204 may be bonded, via a retaining epoxy 230, to either the transducer 208 or to the first conductive medium, such as a gold coating, etc. (not shown.) Naturally, the retaining epoxy 230 should be selected to minimize any interference to the source or return signal. Examples of the retaining epoxy 230 include Epotech 301, Epotech 353, Epotech 377, provided by Epoxy Technology, Inc., Bellerica, Mass. As illustrated in FIG. 7A, the retaining epoxy 230 may run along the sides of the transducer assembly 202 in which case the epoxy 230 may adhere to the elongate member (not shown.) Moreover, the tip 204 may be machined, etched, etc., to contain a plurality of small grooves 232 for seating the retaining epoxy 230. Such a configuration increases the retention strength of the tip 204 within the device and is shown in FIG. 7B which illustrates a magnified view of the section marked 7B found in FIG. 7A. Although not shown, the epoxy 230 may be placed on a lip 234 of the lens 204. In such cases, the epoxy 230 may also adhere to a front end of the elongate member (not shown.)

FIG. 7C illustrates another variation where the tip 204 has a single groove 246 for better retention of the tip 204 in the device. It is noted that the grooves discussed herein may either extend around the entire perimeter of the tip 204 or they may extend over only portions of the tip 204. In the latter case, the term ‘groove’ is intended to include structures such as: dimples, furrows, indentations, pockets, notches, recesses, voids, etc. For sake of illustration, the elongate member is not illustrated in these figures.

FIG. 7D illustrates a variation of a tip 204 having at least one rib 248 which may provide a friction fit with the elongate member 218. The rib 248 may be deformable or rigid.

FIG. 7E illustrates another variation where the tip has at least one grove 246 where the elongate member 218 is either crimped or filled into the groove 246. The elongate member 218 may also be reformed using heat such that it forms/flows into the groove 246.

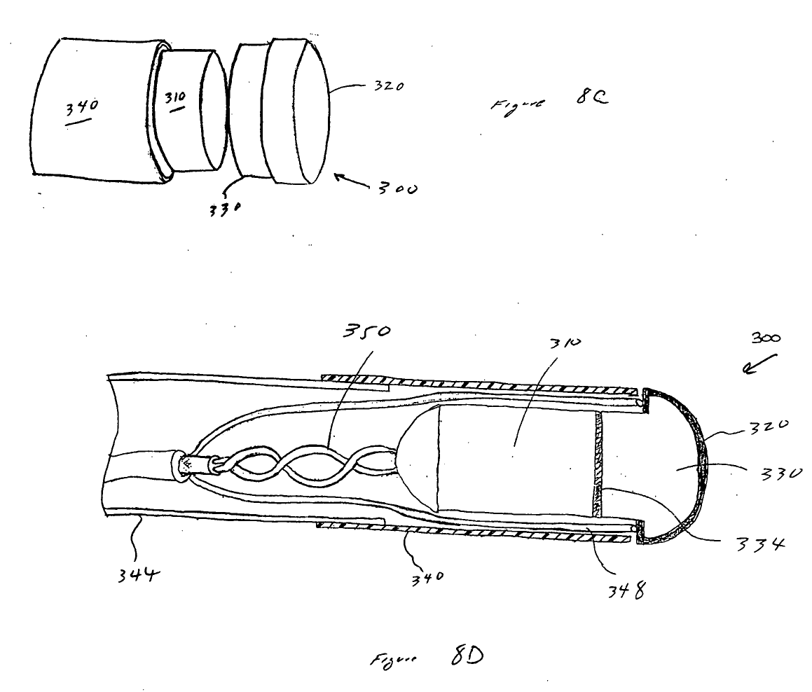

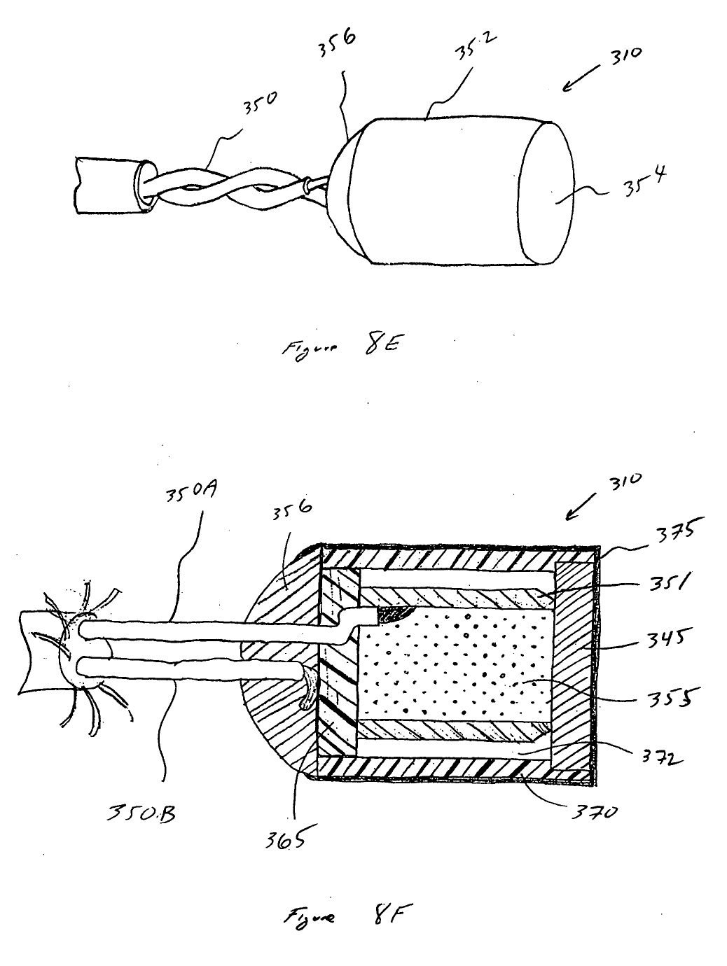

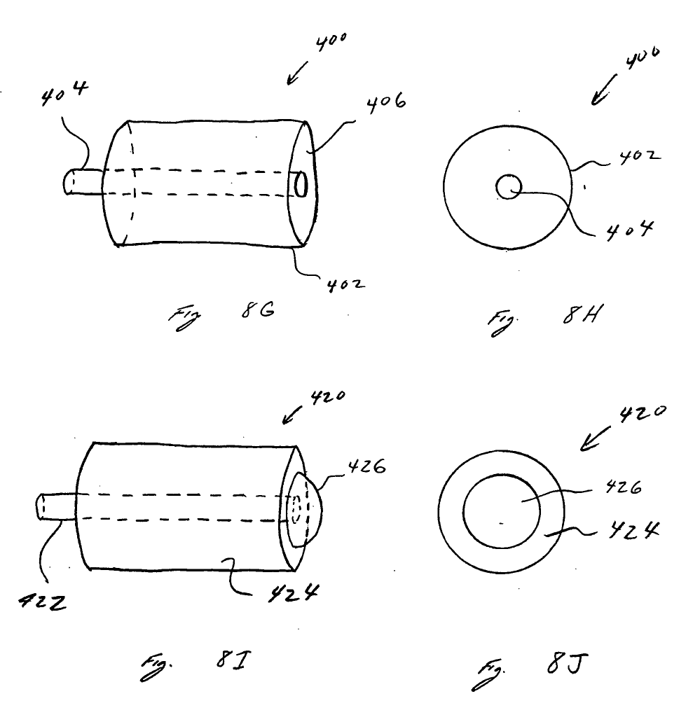

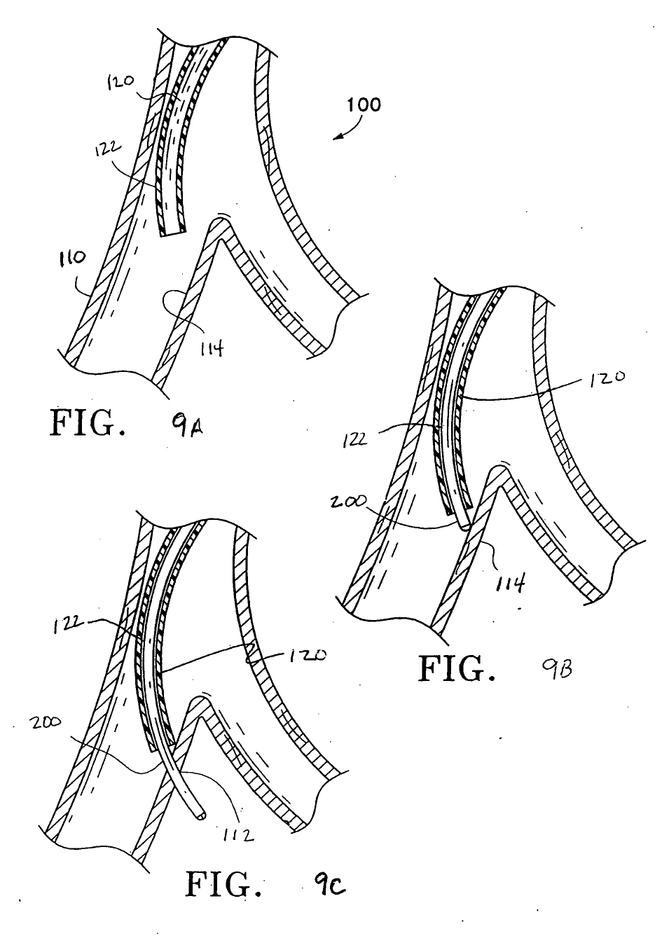

FIGS. 8A and 8B illustrate a medical device having a multifunctional tip assembly. FIG. 8A is an exploded view of a distal section of a medical device or catheter 300 and FIG. 8B is a cross sectional view of a distal section of a catheter 300. The tip section shown in FIG. 8B is assembled and includes additional components not shown in FIG. 8A such as, for example, electrical wires and adhesives.

Referring to FIGS. 8A-8B, the distal section of the catheter 300 includes an ultrasonic transducer 310 for emitting and detecting ultrasonic signals. The ultrasonic transducer may be an assembly as described herein. The ultrasonic transducer 310 serves to sense, e.g., the presence of nearby blood vessels based on a Doppler shift in the reflected ultrasonic signals as discussed above. If no blood vessels are present at the target site, a collateral channel may be created through the tissue by applying RF energy to the tissue via electrode 320. Only a single monopolar type electrode is shown in FIG. 8A. However, as described above, the tip may be configured to have multiple electrodes and may operate in bi-polar or monopolar configurations. The electrode may be ring-shaped and fastened or otherwise attached to the tip. For example an epoxy 332 may be used to attach the electrode 320 to a substrate material 330. The epoxy 332 is preferably able to withstand high temperatures. An example of a suitable epoxy is Masterbond EP42HT.

The substrate material 330 may be thermally and or electrically insulating. That is to say, the substrate material may be selected to thermally and or electrically insulate the ultrasonic transducer 310 from the electrode 320. This serves to reduce noise during ultrasonic detection. Accordingly, an electrically nonconducting material such as ceramic may be a suitable substrate material. However, other materials may be utilized for the substrate material such as, without limitation, glass, aerogel, and other materials that have low acoustic impedance; that are electrically insulating; and/or that are thermally insulating.

The substrate material 330 may be bonded to the transducer with an adhesive 334. The epoxy or adhesive preferably may withstand high temperatures such as 150 to 250 degrees Celsius. Also, the coefficient of thermal expansion for various components of the tip assembly may be closely matched such that stress forces at the interface do not cause the joint to crack or otherwise fail. The materials are preferably biocompatible as well as capable of withstanding sterilization. Also, the substrate material 330 may be fused directly to the transducer assembly with heat and pressure. For example, the outer surface of the transducer assembly may comprise a metal or another type of material which may be heated to fuse the components together. An adhesive may not be necessary in such a case.

FIG. 8B shows a conducting member or wire 348 electrically connected with the electrode 320. The conducting member 348 is connected with an RF controller or generator to supply RF energy to the electrode 320 for tissue ablation as described above. A second conducting member is shown (not numbered) that may serve as a redundant path to supply RF energy to the tip electrode in the event the first wire is damaged or detaches from the tip electrode. Additional conducting members 350 are provided to connect the ultrasonic transducer to an ultrasonic controller. Further details of the ultrasonic assembly 310 are provided below with reference to FIGS. 8E and 8F.

FIG. 8B also shows an elongate member 340 coaxially surrounding the ultrasonic transducer assembly 310. The elongate member may be a flexible cylindrical body or hollow member which is positioned coaxially around the ultrasonic transducer 310 and the substrate material 330 such that it mates with the edge of the electrode 320. The elongate member may extend to the proximal section of the catheter or it may be bonded to a sleeve member 344 as shown in FIG. 8B. The elongate tubular member may be, for example, polyimide or another biocompatible polymer. An epoxy 346 may also be deposited within the tubular body to secure the wires, transducer, and other components in place. Accordingly, a low profile catheter provides ultrasonic detection as well as RF tissue ablation. Furthermore, the diameter of the distal section may be relatively uniform and range from about 1 to 3 mm and perhaps about 1.5 to 2.0 mm and more preferably about 1.8 mm which allows the catheter to be manipulated through small airways, bronchioles, or various deployment instruments.

FIGS. 8C and 8D illustrate another medical device having a multifunctional tip assembly. FIG. 8C is an exploded view of the distal section of a medical device or catheter 300 and FIG. 8D is a cross sectional view of a distal section of a catheter 300. The assembly shown in FIG. 8D is assembled and includes additional components not shown in FIG. 8C such as, for example, the electrical wiring and adhesives.

The multifunctional tip shown in FIGS. 8C and 8D, like the assembly shown in FIGS. 8A and 8B, includes an ultrasonic transducer 310, an electrode 320, and a substrate material 330. However, in the distal section of the catheter shown in FIGS. 8C and 8D, the electrode 320 is a coating of metal disposed on the substrate material 330. The electrode 320 may be disposed on the substrate material by sputtering or any other technique which secures the metal to the substrate. A wide variety of metals may be used including, for example, titanium, titanium alloy, or gold, etc.

Again, a conducting member or wire 348 is electrically connected with the electrode 320. The conducting member 348 is connected with an RF controller or generator to supply RF energy to the electrode 320 for tissue ablation as described above. A second conducting member is shown (not numbered) that may serve as a redundant path to supply RF energy to the tip electrode in the event the first wire is damaged or detaches from the tip electrode. Additional conducting members 350 are provided to connect the ultrasonic transducer to an ultrasonic controller. Further details of the ultrasonic assembly 310 are provided below with reference to FIGS. 8E and 8F. Accordingly, a low profile catheter 300 can provide ultrasonic detection as well as RF tissue ablation. It is also noted that ultrasonic energy may be propagated through the electrode 320, the same electrode that is used for delivering RF energy to heat adjacent tissue. This configuration has the advantage that its diameter or profile may be minimal since the components are axially aligned rather than in a side by side arrangement.

An enlarged perspective view and cross sectional view of the transducer assembly 310 mentioned in FIGS. 8A-8D is shown in FIGS. 8E and 8F respectively. Referring to FIG. 8F, a transducer assembly 310 includes a transducer element 345 which is typically a piezoelectric material. The proximal surface of the transducer element 345 is in electrical communication with a metal conductor such as a metal hypotube 351. A backing layer 355 of epoxy may be present within the hypotube 351 to absorb ultrasonic signals that are generated or transmitted from the proximal surface of the transducer element towards the proximal end of the catheter. An example of an epoxy which is a suitable backing layer is Hysol 2039 and 3561, Loctite Corporation, Rocky Hill, Conn.

A first conducting member 350A or wire is shown electrically connected to the hypotube through an insulating layer 365. The insulating layer 365 may be an insulating epoxy such as, for example, Hysol 2039 and 3561, Loctite Corporation, Rocky Hill, Conn. Additionally, a polymeric tube 370 such as polyimide tubing coaxially surrounds the hypotube 351 to insulate the hypotube. An air gap 372 may be created between the hypotube and the polymeric tube 370. The polymeric tube insulates the hypotube 351 from an electrically conducting metal coat 375 that surrounds the entire distal portion of the transducer assembly including the distal face of the transducer element 345. The metal coat 375 is shown as a relatively thick coating for illustration only. The coat may be a thin coat of sputtered metal such as a gold coat 1-5 angstroms in thickness or a relatively thick layer of metal up to upwards of 3 mm in thickness.

A second conducting member 350B may be electrically connected to the metal coating 375 via a conductive epoxy 356 which is deposited and forms the proximal end of the transducer assembly. An example of a conductive epoxy is silver epoxy or Tra-Con BA-2902, Tra-Con, Inc., Bedford Mass. Also, the conductive epoxy 356 is shown as a hemispherical shape. However, the shape may vary.

The entire transducer assembly may be inserted in an elongate member as shown in FIGS. 8A-8D. In operation, an ultrasound controller (not shown) may be connected with the first and second conducting members 350A, B to create an ultrasonic signal that is emitted through the distal end of the catheter. In particular, the ultrasonic signal emits from the transducer element, propagates through the substrate material and through the distal end. In the catheter shown in FIGS. 8C-8D the ultrasonic signal also propagates through the electrode. Signals reflected off various tissue media are transmitted back through the distal end, through the substrate material and to the transducer element. When it is desired to create a hole or channel through tissue (e.g., an airway wall) as discussed above, the electrode 320 is activated by transmitting a radio frequency signal from a generator (not shown) through a third conducting member or wire 348 to the electrode.

Another distal tip assembly 400 for a catheter is shown in FIGS. 8G-8H. FIGS. 8G and 8H respectively show a partial perspective and front view of an ultrasonic transducer 402 having a cylindrical shape. The transducer 402 shows a bore extending along a center axis of the transducer. However, the bore may extend through the transducer body along a different path. For example, the path may be linear and off-center or the path may be nonlinear.

FIGS. 8G and 8H also show a wire electrode member 404 extending through the bore to the distal end 406 of the assembly 400. The assembly end 406, as shown, may be flat and uncovered. Thus the distal face of the ultrasonic transducer 402 may make direct contact with tissue to be treated. This allows ultrasonic signals to be transmitted directly into the tissue. The signals do not have to pass through additional materials such as a metal electrode or air gaps. This design can serve to minimize signal losses.

FIGS. 8I and 8J depict another distal tip catheter assembly 420 having a center electrode 422 extending axially through an ultrasonic transducer 424. FIG. 8I is a partial perspective view and FIG. 8J is a front view. The assembly shown in FIGS. 8I and 8J is similar to that shown in FIGS. 8G and 8H except that it additionally includes a shaped electrode tip or end 426. As shown, the shaped tip is rounded and hemispherical. The shape of the tip is selected to optimize a desired application such as RF ablation to create a channel through an airway wall. Additional electrodes (e.g., rings) may be incorporated onto the end to provide a bi-polar or monopolar configuration. The rounded electrode tip may be formed a number of ways such as, for example, fusing or otherwise adhering a metal piece to the central electrode 422. The electrode 422 and tip 426 may be an integral component fabricated by conventional machining techniques and then inserted into the bore of the transducer. Still other techniques as is known in the art may be employed to fabricate the distal tip assembly 400.

The diameter of the ultrasonic transducer 402, 424 in the distal section typically ranges from 0.5 to 3 mm and the diameter of the electrode conductor 404, 422 extending through the bore may be much less than this diameter. When a tip is present and protrudes from the end of the transducer face, the diameter of the electrode tip (e.g., tip 426) may range from 0.2 to 2.6 mm and perhaps, between 0.4 and 1.8 mm. Also, the diameter of the electrode tip may be equal to the diameter of the central electrode extending through the bore of the transducer which may be less than 0.4 mm.

In the tip assemblies described in FIGS. 8G-8J the electrode and the ultrasonic transducer may be electrically separated such that passing a current through one component does not activate or interfere with the other. Examples of such insulation include, without limitation, polymeric tubing, epoxies, gaps, etc. Also, the transducer may include a piezoelectric element. A first conducting member is connected to a first face of the piezoelectric element and a second conducting member is connected to a second face such that a current may be delivered across the piezoelectric element to generate ultrasonic signals. A third conducting member may be electrically connected with the central electrode to supply RF current to the electrode and shaped distal tip. Each of the conductive members may be connected to an appropriate controller.

The whole assembly may be placed inside a distal section of an elongate tubular member such that the end of the transducer assembly is flush with the end of the elongate member. Also, the transducer assembly may be inserted and affixed in the elongate member such that at least a portion of the transducer assembly extends beyond the end of the elongate member. Again, the distal tip assemblies shown in FIGS. 8G-8J serve to deliver ultrasonic signals directly into target tissue.

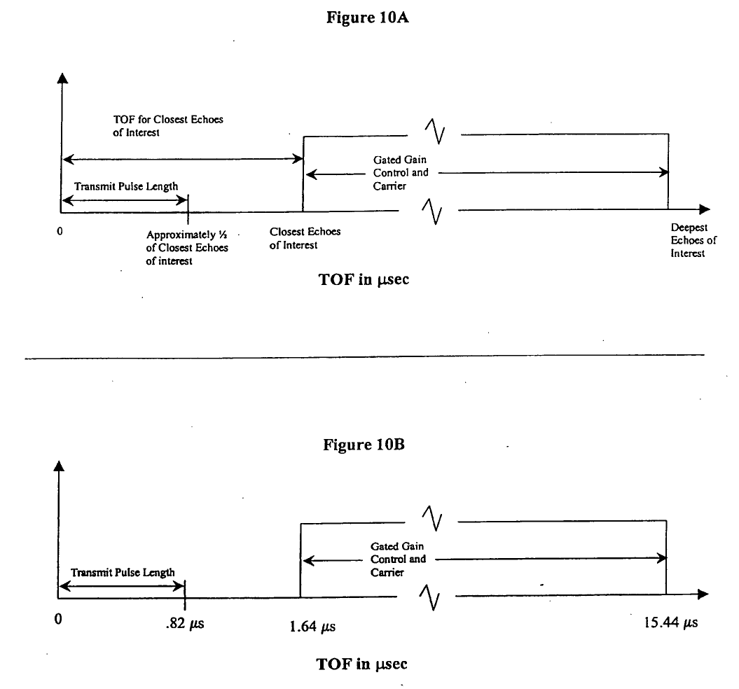

FIGS. 9A-9C illustrate use of the device described above to create a channel through an airway wall of lung tissue. FIG. 9A illustrates the advancement of an access device 120 into the airways 100 of a lung. The access device may be a bronchoscope, endoscope, endotracheal tube with or without vision capability, or any type of delivery device. The access device 120 will have at least one lumen or working channel 122. The access device 120 will locate an approximate site 114 for creation of a collateral channel. In cases where the access device 120 is a bronchoscope or similar device, the access device 120 is equipped so that the surgeon may observe the site for creation of the collateral channel. In some cases it may be desirable for non-invasive imaging of the procedure. In such cases, the access device 120 as well as the other devices discussed herein, may be configured for detection by the particular non-invasive imaging technique such as fluoroscopy, “real-time” computed tomography scanning, or other techniques being used.

FIG. 9B illustrates a variation of the inventive device 200 advanced through the lumen 122 of the access device 120 towards the site 114. An ultrasound signal may be emitted into the tissue. The reflected signals are detected and if a Doppler shift is not present, the site may be a suitable location to create a channel in the airway wall. Again, sensing a Doppler shift can determine whether a blood vessel is adjacent to the site.

FIG. 9C illustrates the creation of a collateral channel 112. As shown in FIG. 9C, the device 200 may be manipulated to a position that is optimal for creation of the collateral channel 112. For example, RF energy may be emitted from the tip of the device 200 to create the channel. Also, little or no force or pressure is required to penetrate the airway wall while RF energy is being delivered to create the channel 112. It is noted that either the access device 120 or the inventive device 200 may be steerable. Such a feature may assist in the positioning of any of the devices used in the inventive method. Although it is not illustrated, as discussed herein, it is desirable to create the collateral channel such that it is in fluid communication with an air-sac. The fluid communication allows for the release of trapped gasses from the hyper-inflated lung.

The inventive device is configured to communicate with an analyzing device or control unit 190 (e.g., see FIG. 2A) adapted to recognize the reflected signal or measure the Doppler shift between the signals. As mentioned above, the source signal may be reflected by changes in density between tissue. In such a case, the reflected signal will have the same frequency as the transmitted signal. When the source signal is reflected from blood moving within a vessel, the reflected signal has a different frequency than that of the source signal. This Doppler effect permits determination of the presence or absence of a blood vessel within tissue. The device may include a user interface which allows the user to determine the presence or absence of a blood vessel at the target site. Typically, the user interface provides an audible confirmation signal. However, the confirmation signal may be manifested in a variety of ways (e.g., light, graphically via a monitor/computer, etc.)

Although depicted as being external to the device 200, it is contemplated that the analyzing device 190 may alternatively be incorporated into the device 200. The transducer assembly of the invention is intended to include any transducer assembly that allows for the observation of Doppler effect, e.g., ultrasound, light, sound etc.

In variations of the invention using pulsed Doppler, the selection of the tip length, as discussed above, sets a parameter for design of the Doppler pulse length and range gate so that excessive echo signal clutter caused by the use of a tip (i.e., a tip capable of emitting and receiving ultrasonic signals and delivering RF energy) is reduced before the arrival of the echo signals from the area of interest.

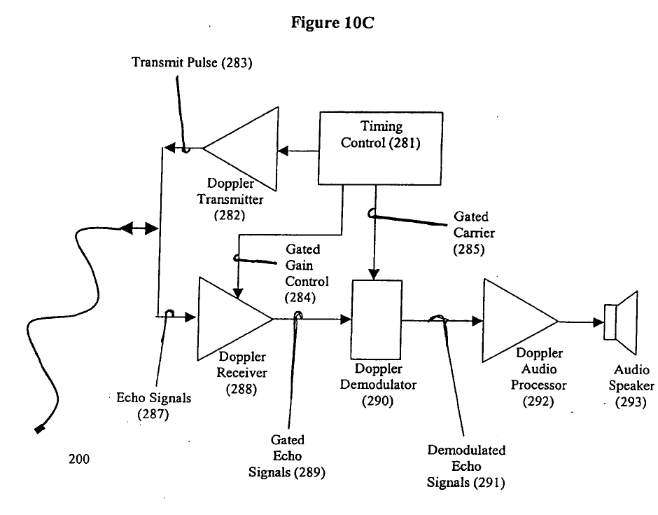

The transmit pulse length may be set to less than the acoustic travel time for an echo signal from the area of tissue to be inspected. This setting allows the receiver to begin recovery from the transmit pulse before the first echo signal arrives at the transducer. As shown in FIG. 10A, the gated gain control and carrier can be set based upon the time-of-flight (TOF) of a signal given pre-desired depths at which the device listens for blood vessels.

The values discussed herein are intended to serve as examples only with the underlying calculations being intended to show the methodology used for Doppler detection of blood vessels. For example, during trials it was found that an acceptable minimum and maximum depth of penetration of the device was 0.8 mm and 10 mm respectively. It is noted that depths are often measured as being normal to the surface of the tissue, and because the device will often approach the tissue at an angle to the surface of the tissue, the maximum and minimum ranges Rₘₐₓ and Rₘᵢₙ used for determining the TOF are adjusted to reflect the normal distance from the tip of the device to the desired depth. (e.g., assuming a 60 degree angle of incidence, and a minimum and maximum depths of 0.8 mm and 10 mm, Rₘᵢₙ=0.92 mm and Rₘₐₓ=11.55 mm.)

The time for a signal to travel from the tip to and from Rₘᵢₙ equals 2Rₘᵢₙ/Cₜᵢₛₛᵤₑ where Cₜᵢₛₛᵤₑ equals the speed of the signal in tissue (approximately 1540 m/s). The time for a signal to travel back and forth through the tip (assuming a 1.33 mm titanium tip, with Cₜᵢₜₐₙᵢᵤₘ=6100 m/s) was found to be 0.44 μs. Therefore, the time for the closest echoes of interest is approximately 1.2 μs plus 0.44 μor 1.64 μs. The Transmit Pulse Length is then set to be less than time for the closest echoes of interest, preferably about ½ of 1.64 μs or ˜0.82 μs. Setting the Transmit Pulse Length to be less than the time for the closest echoes of interest allows the receiver to begin recovery from the reverberation of the transmit pulse in the tip before the first echo signals arrives back at the transducer. As a result, the controller is configured to listen for the first Doppler echo signal starting at the earliest time the first echo signal will return. Based upon the above example, this time is 1.64 μs.

Using a combination of a gated gain control applied to the receiver and a gated carrier applied to the demodulator, the Doppler echo signals are thereafter received until a time that echoes return from the deepest area of interest (e.g., as noted above, 10 mm). This value is calculated based upon the TOF from the tip to the deepest area of interest (15 μs, calculated from 2Rₘₐₓ/Cₜᵢₛₛᵤₑ.) plus the TOF through the tip (0.44 μs as discussed above.) Accordingly, Doppler signals from tissue of up to 1 cm of depth (Rₘₐₓ) may be received up to 15.44 μs. FIG. 10B illustrates the above calculated values as applied to the TOF diagram. As noted above, these values are intended to be exemplary and illustrate the methodology used in determining the timing for the Doppler system. Accordingly, these values may also be adjusted depending upon the desired depth to be examined.

FIG. 10C illustrates an example of a schematic representation of a pulsed wave Doppler electronic system for use with the inventive device. The electronics system uses standard circuit elements.

As illustrated, the timing control 281 supplies timing and control signals to the Doppler transmitter 282, the Doppler receiver 288, and the Doppler demodulator 290. The Doppler transmitter 282 amplifies an applied signal applied to generate a transmit pulse which is ultimately applied to the device 200. In one example, the transmit pulse had a center frequency of 8 MHz and a pulse length of approximately 1 μs and an amplitude of 15 V peak. The transducer at the distal tip of the device 200 converts the transmit pulse into an acoustic pulse. As the acoustic pulse travels through the tissue and blood the structures and cells produce reflections that travel back toward the probe tip. The reflections are converted from acoustic echoes to electrical echo signals 287. These echo signals 287 consist of a mixture of signals, some of a frequency equal to that of the transmitted signal (echoes from stationary structures in the ultrasonic field), and some echoes that are shifted in frequency by the Doppler effect. The echo signals 287 are amplified by the Doppler receiver 288. A gated gain control 284 is set to start increasing gain after the transmit pulse ends but soon enough for echo signals 287 of interest to be amplified. The gated gain control 284 lasts until echo signals 287 from the deepest structures of interest have been amplified. These echo signals 287 are demodulated in the Doppler demodulator 290 using a gated carrier 285 in order to produce demodulated echo signals 291 that contain Doppler signals from moving blood cells at audio frequencies. The demodulated echo signals 291 are then filtered and amplified by the Doppler audio processor 292 to improve the signal fidelity of the Doppler audio signals. These filtered and amplified signals are then sent to the Audio Speaker 293.

It should be noted that the wires shown in the various embodiments may include an insulation or shield to prevent electrical current from passing from one wire to another component. It is also noted that the device may also be designed to have a double shield. First, the twisted pair wires connecting the transducer assembly to the Doppler control unit 190 will be shielded. Furthermore, because the energy supply 188 may be delivered through one of the pair of wires, the outer portion of the catheter that is exposed proximal to the working channel of an endoscope will also be shielded to prevent undesirable conduction of current.

All publications, patent applications, patents, and other references mentioned above, and hereinafter, are incorporated by reference in their entirety. To the extent there is a conflict in a meaning of a term, or otherwise, the present application will control.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the invention.

1. A medical catheter having a proximal section, a distal section and a distal end, said catheter comprising:
an ultrasonic transducer positioned in said distal section, said ultrasonic transducer adapted to emit and receive ultrasonic signals; and
a tip assembly located at and forming said distal end of said catheter, said tip assembly comprising an acoustically-transmitting material and an electrode at least partially coating said acoustically-transmitting material, said acoustically-transmitting material insulating said electrode from said ultrasonic transducer, wherein said electrode and said ultrasonic transducer are positioned such that when ultrasonic signals are emitted and received by said ultrasonic transducer said signals are transmitted through said electrode.

2. The catheter of claim 1 wherein said acoustically-transmitting material is ceramic.

3. The catheter of claim 1 wherein said electrode is an electrically-conductive coating partially covering said acoustically-transmitting material.

4. The catheter of claim 1 wherein said acoustically-transmitting material is adjacent to a distal surface of said ultrasonic transducer.

5. The catheter of claim 4 wherein said acoustically-transmitting material is adhered to the distal surface of said ultrasonic transducer.

6. The catheter of claim 3 comprising a flexible tubular member coaxially surrounding said ultrasonic transducer.

7. The catheter of claim 6 wherein said tubular member extends partially over said acoustically-transmitting material such that a distal edge of said tubular member mates with a proximal edge of said electrode coating.

8. The catheter of claim 1 wherein said ultrasonic transducer is a piezoelectric transducer.

9. The catheter of claim 4 wherein said acoustically-transmitting material is fused to the distal surface of said ultrasonic transducer.

10. The catheter of claim 1 further comprising an electrode-conducting element connected to said electrode and wherein said electrode-conducting element is electrically insulated from said ultrasonic transducer.

11. The catheter of claim 1 wherein said acoustically-transmitting material has an axial length less than 0.9 mm.

12. The catheter of claim 1 wherein said tip assembly has a flat end.

13. The catheter of claim 1 wherein said distal section has a constant outer diameter.

14. The catheter of claim 8 wherein said ultrasonic transducer comprises a conductive coating disposed over at least a portion of a distal surface of an ultrasonic transducer element.

15. The catheter of clam 14 wherein said ultrasonic transducer comprises a metal conductive member in electrical contact with a proximal surface of said ultrasonic transducer element.

16. The catheter of claim 15 comprising a first conductive wire in electrical contact with said metal conductive member.

17. The catheter of claim 16 wherein said metal conductive member is a metal tube.

18. The catheter of claim 14 comprising a second conductive wire in electrical contact with said conductive coating disposed over at least a portion of a distal surface of the ultrasonic transducer element.

19. The catheter of claim 17 further comprising a backing layer within said metal tube, said backing layer made of material which does not acoustically transmit ultrasonic waves.

20. The catheter of claim 1 wherein said acoustically-transmitting material electrically insulates said electrode from said ultrasonic transducer.
